(19) United States
(12) Patent Application Publication
(10) Pub. No.: US 2026/0301871 A1
(43) Pub. Date: Oct. 1, 2026

(54) METHODS AND SYSTEMS FOR DESIGNING LONGEVITY-ASSOCIATED NUCLEIC ACID SEQUENCES USING DNARP

(71) Applicant: Jonathan Washburn, Austin, TX (US)

(72) Inventor: Jonathan Washburn, Austin, TX (US)

(21) Appl. No.: 19/629,612

(22) Filed: Mar. 26, 2026

Related U.S. Application Data
(60) Provisional application No. 63/778,386, filed on Mar. 27, 2025.

(51) Int. Cl. G16B 30/20; C12N 15/85

(52) U.S. Cl. CPC G16B 30/20; C12N 15/85

(57) ABSTRACT

A computer-implemented method, system, and platform for selecting nucleic acid sequences for age-reversal-associated use are disclosed. Candidate sequences associated with a longevity-associated target are generated or received and evaluated using a DNA Recognition Physics framework D=(S, H, E), where S represents sequence stability, H represents helical structural configuration, and E represents coherence-energy selection. A sequence stability score is computed and compared to a stability threshold, structural compatibility is evaluated under a selected helical parameter set, and a coherence level is selected from an allowed set. A predicted enhancement score is then computed from the sequence stability score and the selected coherence level, and candidate sequences are ranked and selected for synthesis, construct assembly, editing, delivery, validation, or therapeutic use. The disclosed framework supports regulatory sequences, coding sequences, donor templates, multi-gene cassettes, and delivery-ready payloads for longevity, repair, and age-reversal-associated applications.

### CROSS-REFERENCE TO RELATED APPLICATIONS

This application claims benefit of priority to U.S. Provisional Patent Application No. 63/778,386, titled “Method for Enhancing DNA Sequences for Age-Reversal Using DNARP (DNA Recognition Physics)” filed on Mar. 27, 2025.

### BACKGROUND

This invention relates to biotechnology, synthetic biology, genetic engineering, computational biology, and age-reversal therapeutics. More particularly, it relates to computer-implemented methods, systems, and design workflows for generating, evaluating, selecting, synthesizing, and using nucleic acid sequences, including DNA constructs and related therapeutic or research compositions, using DNA Recognition Physics (DNARP) to improve longevity-associated function, genomic stability, repair capability, and age-reversal performance. Biological aging is associated with a constellation of molecular and cellular changes, including telomere shortening, epigenetic drift, accumulation of somatic mutations, mitochondrial dysfunction, impaired proteostasis, impaired autophagy, dysregulated nutrient sensing, and declining genome maintenance and repair. These features contribute to reduced cellular resilience, increased senescence-associated phenotypes, and progressive loss of tissue and organismal function. There is accordingly substantial interest in interventions capable of increasing longevity-associated gene output, enhancing repair-associated pathways, supporting telomere maintenance, and reducing one or more indicators of biological aging.

Existing approaches, however, remain subject to substantial limitations. Empirical pharmacologic approaches, including NAD+boosters, rapamycin, metformin, and senolytics, are typically discovered and optimized without a first-principles sequence-design framework and can be highly context-dependent in dosing, timing, and genetic background. Gene-therapy and telomere-extension approaches can deliver known targets, but generally do not provide a computational method for designing sequence architecture itself to improve stability, structural compatibility, and age-reversal-associated performance. Likewise, current CRISPR-based and other editing approaches focus primarily on targetability or correction of a locus rather than first-principles optimization of sequence stability, helical structure, and coherence-related transcriptional performance, while broad epigenetic reprogramming approaches can be difficult to control and may lack a quantitative sequence-level design rule. AI- and machine-learning-based sequence-design methods can also be model-dependent and difficult to interpret mechanistically. Accordingly, there remains a need for a computationally grounded design framework capable of generating and ranking DNA sequences for age-reversal applications using explicit, reproducible design parameters and objective acceptance criteria.

### SUMMARY

The disclosed DNARP workflows improve computer-implemented sequence design by applying an ordered set of objective gating operations—stability-threshold filtering, helical structural compatibility filtering using an explicitly parameterized model and tolerance criterion, and coherence-level selection from an allowed set—to reduce search space and computational waste and to produce a ranked output set suitable for downstream synthesis, construct assembly, editing, delivery, and validation. In representative implementations, the system generates or updates a candidate record in a data store with the computed stability score, structural evaluation result, coherence selection, and predicted enhancement score, and outputs a ranked results report for use in manufacturing and validation workflows.

In some embodiments, the invention provides computer-implemented methods, systems, and design workflows for generating, evaluating, selecting, synthesizing, assembling, editing, delivering, validating, and using nucleic acid sequences for age-reversal-associated applications using a DNA Recognition Physics (DNARP) framework. In some embodiments, the framework applies a program D=(S, H, E), where S represents sequence stability optimization, H represents helical structural optimization, and E represents coherence-energy optimization. Candidate nucleic acid sequences can be generated or received for a selected longevity-associated target, evaluated according to one or more sequence-stability, structural, and coherence-related criteria, ranked according to a predicted enhancement score or related combined score, and then selected for downstream synthesis, construct assembly, editing, delivery, or validation. The invention can be implemented as a computer-implemented method, a design system, a non-transitory computer-readable medium, a sequence-design platform, a method of producing a nucleic acid construct, a method of producing an age-reversal-associated biological effect, or another implementation in which a DNARP-selected sequence is used in research, therapeutic, editing, or validation contexts.

In some embodiments, a candidate sequence is evaluated by computing a sequence stability score, determining whether that score satisfies a stability threshold, evaluating structural compatibility under a selected helical parameter set, selecting a coherence level from an allowed set of coherence levels, and computing a predicted enhancement score based on the stability score and the selected coherence level. In some embodiments, the sequence stability score is computed as a weighted sum of (i) a CG occurrence count and (ii) an AT occurrence count under a stored counting convention, and the stored counting convention is applied consistently across candidates within a ranking run. In some embodiments, coherence selection is used to place a candidate sequence into a selected energetic or output regime that influences predicted enhancement and ranking. Candidate sequences satisfying the applicable thresholds and ranking criteria can be selected as individual regulatory sequences, coding sequences, donor templates, repair templates, multi-gene cassette elements, assembled constructs, or delivery-ready payloads.

In some embodiments, the invention is applied to a target associated with one or more longevity-associated genes, including for example SIRT1, FOXO3, TERT, XRCC1, RAD51, ATM, PARP1, or KLOTHO. In one illustrative embodiment, a SIRT1-associated target region is used to generate a library of candidate regulatory sequences, the candidate regulatory sequences are screened according to sequence stability, structural compatibility, and coherence selection criteria, and one or more top-ranked candidates are selected for synthesis and validation as age-reversal-associated regulatory constructs. In another illustrative embodiment, multiple target-associated elements are optimized and assembled into a multi-gene cassette comprising two or more elements associated with longevity, repair, telomere support, or stress-response function, and the resulting construct is selected as a delivery-ready payload for ex vivo, in vivo, or editing-based implementation. In some embodiments, selected outputs are advanced through synthesis, reporter validation, repair assays, senescence or lifespan assays, in vivo assessment, and therapeutic-use workflows, thereby providing a unified platform for moving from computational design to practical age-reversal-associated sequence implementation.

The computer-implemented method and the system embodiments provide a common DNARP selection platform that generates a selected nucleic acid sequence output and associated ranked output records. The construct-production and biological-effect embodiments use that platform output as an input to downstream synthesis, assembly, editing, delivery, and validation operations. Accordingly, the various claim categories are directed to a single inventive concept centered on generating and using DNARP-selected sequences under objective stability, structural, and coherence criteria.

### BRIEF DESCRIPTION OF THE DRAWINGS

The various advantages of the examples will become apparent to one skilled in the art by reading the following specification and appended claims, and by referencing the following drawings, in which:

FIG. 1 is a flowchart showing an overall DNA Recognition Physics age-reversal design method, including target selection, candidate sequence generation or intake, sequence stability scoring, helical structural evaluation, coherence-energy selection, combined scoring and ranking, and synthesis, validation, or implementation of selected candidates.

FIG. 2 is a block diagram showing a DNA Recognition Physics design framework including a sequence stability component, a helical structural component, and a coherence-energy component, together with associated threshold, structural, coherence, and predicted-enhancement parameters.

FIG. 3 is a flowchart showing a sequence stability scoring workflow in which a candidate sequence is received, CG occurrence count and AT occurrence count under a counting convention, a stability score is calculated, and the candidate is accepted or rejected based on a stability criterion.

FIG. 4 is a flowchart and schematic representation showing a helical structural optimization workflow in which a candidate sequence is evaluated according to a pitch parameter, a groove-ratio parameter, a structural compatibility function, and a structural tolerance criterion.

FIG. 5 is a flowchart showing a coherence-energy optimization workflow in which a coherence level is selected from an allowed set, mapped to an associated energy or rate regime, and applied to generate a coherence-adjusted candidate output and a predicted activity-related output.

FIG. 6 is a block diagram showing a DNA Recognition Physics age-reversal simulator system including sequence intake, stability scoring, helical scoring, coherence selection, candidate ranking, construct assembly or export, assay planning or report generation, and candidate data storage functions.

FIG. 7 is a workflow diagram showing an example optimization process for a SIRT1-associated target region, including generation of a candidate library, stability screening, structural screening, coherence selection, candidate ranking, sequence selection, and validation.

FIG. 8 is a workflow diagram showing an example multi-gene optimization process in which multiple longevity-associated or repair-associated elements are selected, arranged into a cassette architecture, assembled into a composite construct, ranked, and prepared as a delivery-ready payload.

FIG. 9 is a diagram showing a candidate ranking and output interface in which candidate records are organized according to stability, structural, coherence, and combined scoring information and separated into accepted and rejected candidate groupings.

FIG. 10 is a validation workflow showing synthesis, reporter testing, repair testing, senescence or lifespan testing, in vivo assessment, control comparison, and pass-fail determination for selected candidates.

FIG. 11 is a therapeutic implementation workflow showing example delivery, editing, ex vivo modification, administration, treatment-response, and follow-up pathways for selected nucleic acid constructs and related payloads.

FIG. 12 is a block diagram showing an example computing system configured to perform the disclosed sequence-design, scoring, ranking, construct-generation, and validation-planning methods.

### DETAILED DESCRIPTION

Unless context requires otherwise, the following applies. “DNARP” refers to the DNA Recognition Physics design framework used to generate, evaluate, rank, select, and implement nucleic acid sequences for longevity-associated and age-reversal-associated applications. A “DNARP program” can be expressed as D=(S, H, E), where S is sequence stability, H is helical structural configuration, and E is coherence-energy configuration. A candidate sequence can be a naturally occurring, engineered, combinatorial, codon-varied, promoter-engineered, donor-template, cassette-derived, or computationally generated sequence and can include DNA or RNA-corresponding embodiments where stated. A target can be a gene, locus, promoter, enhancer, coding region, untranslated region, donor-template region, repair-template region, or other sequence region.

“Longevity-associated gene” includes genes and related targets associated with lifespan, repair, genomic stability, stress response, or telomere biology, including one or more of SIRT1, FOXO3, TERT, XRCC1, RAD51, ATM, PARP1, and KLOTHO. “Age-reversal” includes measurable or predicted reduction, slowing, offsetting, or partial reversal of one or more biological aging features, including increased cellular lifespan, increased repair capacity, reduced senescence-associated markers, and improved age-associated cellular function. “Stability score” includes a computed sequence stability value, including S_stab. “Coherence level” includes a selected setting such as n=1, n=2, or n=3 used for output-state selection. “Pitch” and “groove ratio” refer to helical structural parameters used for structural compatibility evaluation. “Predicted enhancement” refers to a computed value derived from one or more of stability score, structural compatibility, and coherence level. “Therapeutically effective amount” means an amount sufficient to produce a desired biological effect in a cell, tissue, or subject.

“Delivery vehicle” includes viral and non-viral systems including adeno-associated virus, lentivirus, adenovirus, plasmids, minicircle DNA, lipid nanoparticles, polymeric particles, electroporation systems, ribonucleoprotein-associated delivery systems, and ex vivo delivery workflows. “Construct” includes a nucleic acid arrangement comprising one or more coding and/or regulatory elements. “Cassette” includes two or more associated sequence elements configured for coordinated function. “Editing template” includes a sequence for template-guided editing workflows such as homology-directed repair and prime editing. “About” and “approximately” include practical tolerance around stated values. “Comprising” is open-ended. Singular includes plural where appropriate, and disclosed values and equations are non-limiting embodiments unless expressly required by a claim.

The disclosed framework is an explicit, computer-implementable design architecture for selecting candidate nucleic acid sequences. The same framework supports single-sequence and multi-sequence constructs, including promoter, enhancer, coding, donor-template, repair-template, and cassette embodiments.

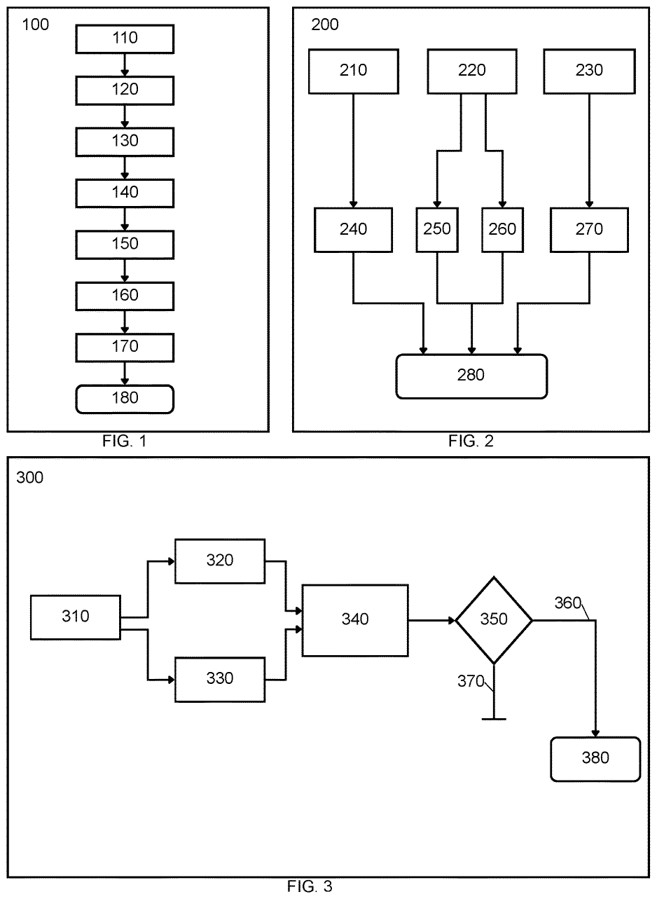

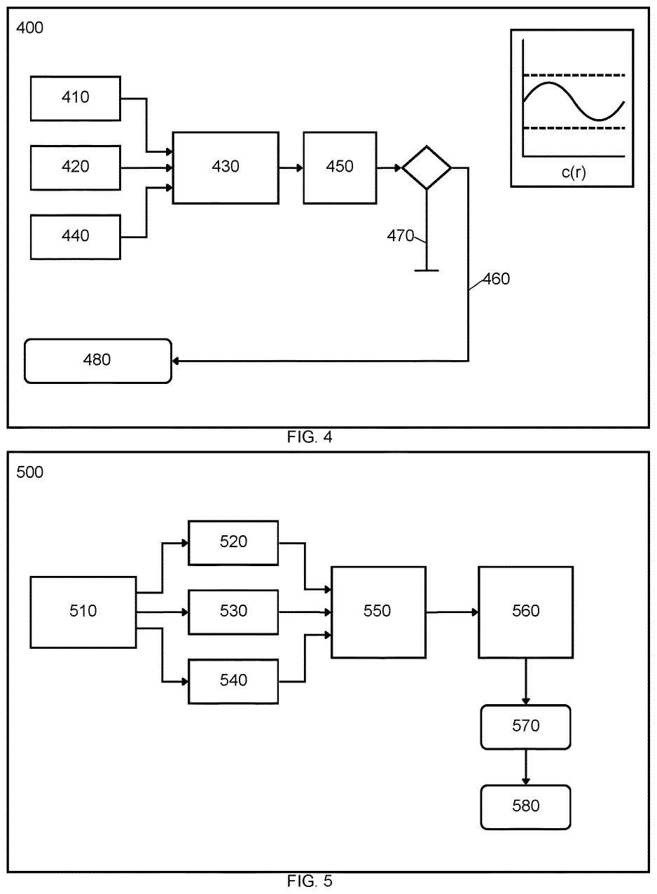

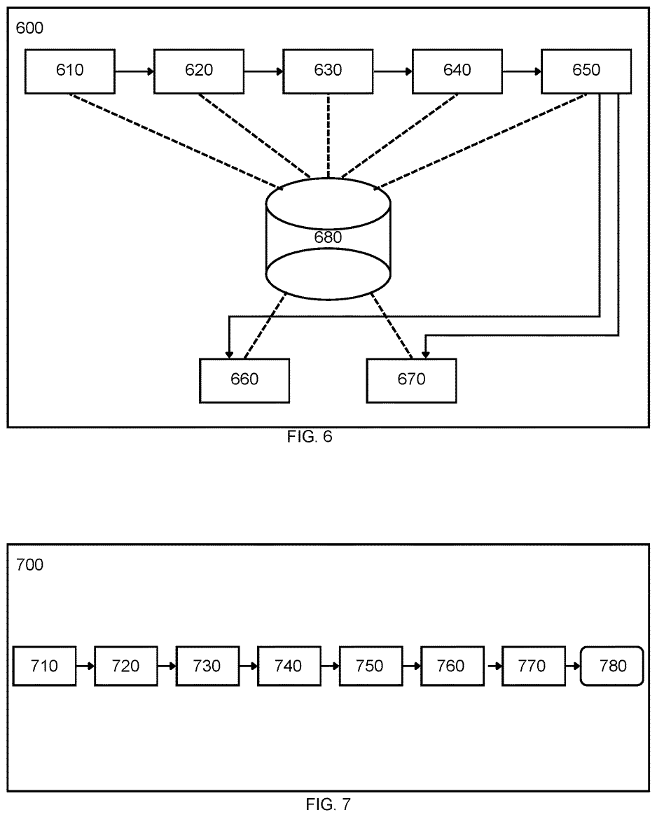

As shown in FIG. 1, workflow (100) includes: target selection (110), candidate sequence generation/intake (120), sequence stability scoring (130), helical structural evaluation (140), coherence-energy selection (150), combined scoring/ranking (160), and synthesis/validation/implementation (170), yielding selected output sequence set (180). As shown in FIG. 2, framework (200) includes sequence stability component (210), helical structural component (220), coherence-energy component (230), stability threshold/criterion (240), pitch parameter (250), groove ratio parameter (260), coherence level parameter (270), and predicted enhancement module/output (280).

In one age-reversal embodiment, parameters are S_stab>=4.5, P=32 A, G=phi, and n=2, with predicted enhancement based on (S_stab/3)\hat{ }2*2. Other disclosed operating points include neighboring ranges and allowed coherence options while preserving D=(S, H, E).

Target selection stage (110) defines biological objective and implementation constraints. Targets can include single-gene, multi-gene, regulatory, coding, or editing objectives and can be chosen manually, semi-automatically, or automatically. Selection inputs can include sequence and locus data, target annotations, expression objectives, cell/tissue context, age-state context, repair objectives, delivery mode, payload size, manufacturability constraints, and editing constraints. Target selection may include inclusion/exclusion criteria and outputs a structured target specification for downstream processing.

Counting convention for #CG and #AT. As used herein, #CG and #AT are computed by scanning the candidate nucleic acid sequence as a string of bases and counting dinucleotide occurrences of “CG” and “AT,” respectively, using an overlapping sliding window of length 2 across positions i . . . i+1 for i=1 to (L−1), where L is sequence length in bases. For avoidance of doubt, each occurrence contributes 1 count, overlapping occurrences are permitted, and ambiguous bases (e.g., “N”) are ignored for purposes of #CG and #AT. In alternative embodiments, #CG and #AT can be computed as mononucleotide counts of bases C+G and A+T, respectively, provided the selected convention is used consistently for all candidates in a ranking run and is stored as metadata with each candidate record.

Candidate sequence generation/intake stage (120) transforms target specifications into analyzable candidates. Candidates can be: naturally occurring references, mutational libraries, combinatorial variants, promoter-engineered variants, codon-optimized coding variants, CRISPR donor variants, homologous recombination templates, concatenated multi-gene cassette variants, base-edited in situ variants, CpG-weighted variants, and RNA-corresponding variants. Candidate generation can be de novo, imported, or hybrid. Candidate intake includes normalization, sequence validation, metadata assignment, and storage in candidate sequence database/data store (680) and/or sequence database (1260), including candidate provenance and status. Sequence stability scoring stage (130) implements S component and can be performed by workflow (300) and/or stability scoring module (620). A canonical embodiment uses:

$$S\_stab = 1.5 * ( \# CG ) + 1. * ( \# AT )$$

where #CG and #AT are counts under an explicitly defined counting convention. The counting convention is explicitly defined herein and, in alternative embodiments, a selected alternative convention is applied consistently across candidates within a ranking run and stored as metadata with each candidate record. In workflow (300), candidate sequence input (310) is processed into CG contribution count (320) and AT contribution count (330), then stability score calculator (340), then threshold comparator (350), producing acceptance path (360) and accepted candidate output (380), or rejection path (370).

A baseline threshold can be 3.0, with an age-reversal threshold of 4.5; stronger optional thresholds include at least 5.0, 6.0, or 7.5. In one explicit rule: accept for downstream evaluation when S_stab>=4.5 and reject when S_stab<4.5. Stability can be global, local-window, normalized, region-specific, or construct-level. Optional variants include methylation-adjusted weighting, position-specific weighting, promoter/coding weighting, motif-preserving weighting, and repair-hotspot weighting. Stability scoring can also be iterative, including redesign and rescoring.

Helical structural evaluation stage (140) implements H component and can be performed by workflow (400) and/or helical scoring module (630). Structural evaluation uses pitch parameter (250)/(410), groove ratio parameter (260)/(420), structural compatibility function (430), evaluation domain/position variable (440), and tolerance window (450), producing structural acceptance output (460), structurally accepted candidate output (480), or structural rejection output (470).

A disclosed structural expression is:

$$C ( r ) = 1 - 0.00567 cos ( 2 pi * r / 32 )$$

which can be evaluated directly or used to derive structural compatibility/deviation metrics. In one embodiment, candidate acceptance requires structural deviation<=0.006 over the selected evaluation domain. Additional disclosed ranges include pitch about 32 A or within 30 A to 40 A (including 31 A to 34 A), groove ratio about phi or within 1.5 to 1.7, and tolerance options including <=0.01, <=0.008, <=0.006, or <=0.005. As used herein, “phi” refers to the golden ratio and is approximately 1.6180339887. When a groove ratio is described as “about phi,” the groove ratio is within a tolerance band around 1.618, for example within 1.5 to 1.7 unless otherwise stated. Structural evaluation can be global or local and can be used both for gating and ranking.

Structural deviation metric and evaluation domain. Structural compatibility is evaluated by computing a deviation metric Dev between an ideal reference profile and a candidate-derived profile over an evaluation domain of r. In one embodiment, r is sampled over an interval r∈[0, P] using step size Δr, where P is the pitch parameter (e.g., about 32 angstroms) and Δr is selected from 0.1 to 1.0 angstroms. The deviation metric Dev can be computed as max_r|C_cand(r)−C_ref(r)|(maximum absolute deviation) or as an RMS deviation, and the candidate satisfies the tolerance criterion when Dev is no greater than the stated tolerance (e.g., ≤0.006).

Sequence-derived candidate profile (example implementation). In one implementable embodiment, the candidate-derived profile C_cand(r) is computed from the candidate nucleic acid sequence by deriving a sequence-dependent feature vector over the sequence and mapping that feature vector to a continuous profile over the evaluation domain. For example, the system: (1) computes a local dinucleotide feature vector f(i) over the candidate sequence using the same stored counting convention applied for #CG and #AT (e.g., overlapping windows over positions i . . . i+1 for i=1 to L−1), optionally including additional dinucleotide classes; (2) maps f(i) to a local sequence-dependent stiffness or curvature proxy k(i) using a stored mapping table; (3) interpolates k(i) to a continuous function k(r) over r in [0, P]; and (4) computes C_cand(r) as a normalized function of k(r) to yield a profile directly comparable to C_ref(r) under the selected deviation metric Dev. The mapping table, interpolation method, and normalization convention can be stored as part of the helical parameter set metadata to support auditability and reproducibility of Dev computations across ranking runs.

Reference profile alignment and storage. In representative embodiments, C_ref(r) is the disclosed analytic expression evaluated over the same domain and step size, and Dev is computed as either a maximum absolute deviation or an RMS deviation over the sampled domain. In one embodiment, Dev is computed as max over r of abs(C_cand(r)−C_ref(r)). In another embodiment, Dev is computed as an RMS deviation over the sampled r values. The system may store, in the candidate record, the selected Dev type (max or RMS), the sampling step size dr, the resulting Dev value, and an identifier for the helical parameter set and mapping table used to generate C_cand(r).

Coherence-energy selection stage (150) implements E component and can be performed by workflow (500) and/or coherence selection module (640). Coherence level set (510) can include first option (520) n=1, second option (530) n=2, and third option (540) n=3. Energy-rate mapping block (550) maps selected coherence state to associated output-state variables, yielding selected coherence state (560), coherence-adjusted candidate output (570), and predicted transcription/activity rate output (580). In some embodiments, coherence-energy selection uses a Recognition-derived coherence quantum E_coh defined as E_coh=phi\hat{ }(−5) in RS-native (dimensionless) units. In such embodiments, a discrete coherence level n is mapped to a coherence energy En using En=n*E_coh, where n is selected from an allowed set (for example n=1, n=2, or n=3). This mapping is used as an implementation option for the E component and does not preclude other energy mappings described herein. In Recognition-native form, E_coh and En are dimensionless RS-native quantities. Any numerical reporting of E_coh or En in electron-volts (eV), joules (J), or other SI/display units is a reporting convention that uses an explicit calibration seam to map RS-native quantities to the selected display units. Accordingly, any eV values stated herein are provided as example reporting values under a declared calibration seam and are not additional fit parameters.

In a disclosed embodiment, n=2 is preferred for balanced age-reversal output and can correspond to an n=2 coherence energy E2 under En=n*E_coh; under an example reporting calibration seam, E2 may be reported as approximately 0.182 eV, with example output-state variables including R=100 bases/s and baseline R0=50 bases/s. n=1 and n=3 are also supported, including stronger-output embodiments at n=3. Coherence selection can be rule-based, user-guided, or automated and can be global or region-specific.

Combined scoring/ranking stage (160) integrates S, H, and E outputs. A disclosed predicted enhancement relation is:

$$Predicted enhancement = ( S\_stab / 3 ) \hat{} 2 * n$$

$$including the specific n = 2 form :$$

$$Predicted enhancement = ( S\_stab / 3 ) \hat{} 2 * 2$$

Predicted enhancement module/output (280), candidate ranking module (650), and candidate ranking/output interface (900) can compute and display candidate record (910) fields including stability score field (920), structural score field (930), coherence field (940), and combined score field (950), and can classify candidates into accepted tier (960) and rejected tier (970), with output report/ranked results report (980).

Ranking can use predicted enhancement alone or multi-factor scoring including one or more of structural conformity, repair score, lifespan score, payload feasibility, manufacturability, off-target constraints, regulatory compatibility, and assay readiness. Candidate selection can use top-N or minimum-score logic, or both. In one explicit selection rule, a candidate is selected when it satisfies: (i) stability threshold, (ii) structural tolerance, (iii) allowed coherence selection, and (iv) ranking criterion (e.g., top-N or minimum combined score).

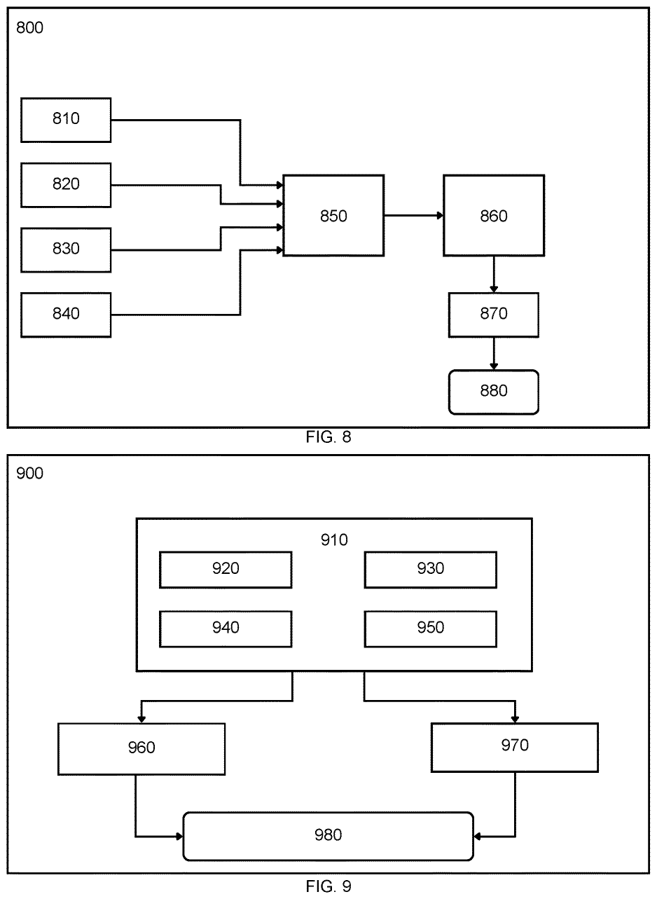

FIG. 7 illustrates a SIRT1 workflow (700): SIRT1 target region (710), SIRT1 variant library (720), stability-passing subset (730), structurally passing subset (740), coherence-selected subset (750), ranked SIRT1 candidate list (760), selected SIRT1 optimized sequence (770), and SIRT1 validation result (780). FIG. 8 illustrates multi-gene workflow (800): SIRT1 cassette element (810), FOXO3 cassette element (820), TERT cassette element (830), repair-gene cassette element (840), linker/spacer architecture (850), assembled multi-gene construct (860), ranked multi-gene candidate set (870), and delivery-ready payload (880).

DNARP age-reversal simulator system (600) can include sequence intake module (610), stability scoring module (620), helical scoring module (630), coherence selection module (640), candidate ranking module (650), construct assembly/export module (660), assay planning/report generation module (670), and candidate sequence database/data store (680). The simulator can run end-to-end workflows, including candidate intake, stability filtering, structural filtering, coherence assignment, ranking, top-N selection, construct export, and validation planning. The simulator can also support iterative redesign when candidates fail at stability, structural, or ranking stages.

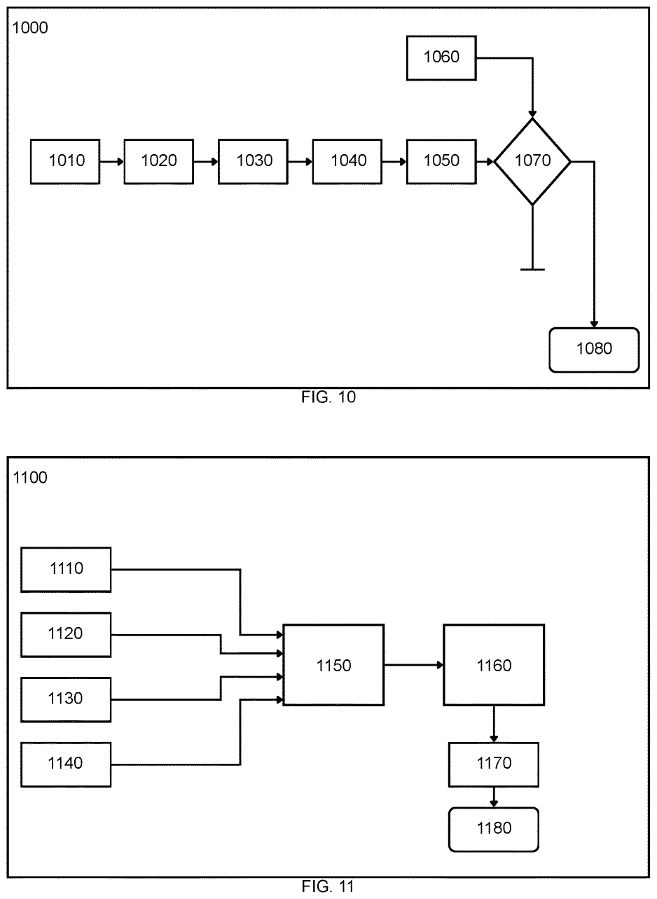

Validation workflow (1000) of FIG. 10 includes synthesis step (1010), reporter assay step (1020), repair assay step (1030), senescence/lifespan assay step (1040), in vivo assessment step (1050), control comparator (1060), pass/fail decision block (1070), and validated construct set (1080). In representative embodiments, pass criteria can include improvement over control by at least 10%, at least 20%, or at least 30%, depending on assay objective.

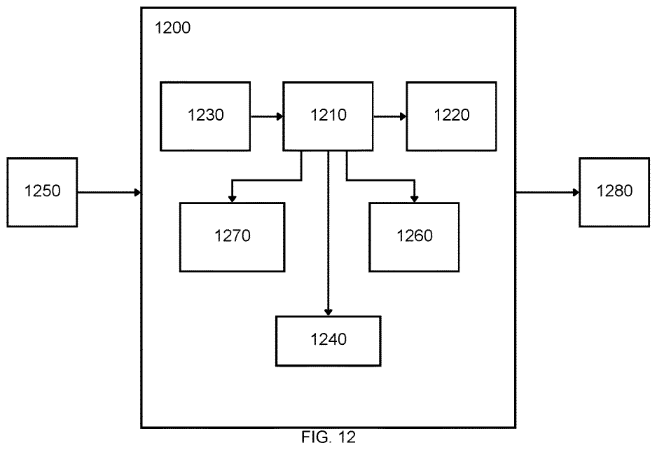

Therapeutic implementation workflow (1100) of FIG. 11 includes viral delivery vehicle (1110), non-viral delivery vehicle (1120), CRISPR editing workflow (1130), ex vivo cell modification workflow (1140), target cell or cell population (1150), subject administration step (1160), age-reversal response/treatment output (1170), and monitoring/follow-up step (1180). Delivery can include viral vectors, plasmids, minicircle DNA, lipid nanoparticles, and other disclosed carriers. Editing can include CRISPR-HDR, base editing, prime editing, and related locus-targeted workflows. Ex vivo and in vivo applications are both supported.

Methods of use include introducing a selected construct or editing payload into cells or administering to a subject to increase expression or activity of longevity-associated targets, increase repair-associated function, reduce biological aging indicators, increase cellular lifespan, or combinations thereof. The target cell population (1150) can include fibroblasts, stem/progenitor cells, immune cells, epithelial cells, neuronal cells, hepatic cells, muscle cells, and other relevant mammalian cells.

Computing system (1200) of FIG. 12 can include processor (1210), memory (1220), storage (1230), network interface (1240), user interface (1250), sequence database (1260), software engine/executable instruction set (1270), and output device/report interface (1280). The processor executes instructions to receive targets/candidates, compute S_stab, apply structural compatibility, select coherence, compute predicted enhancement/combined score, rank and select candidates, and generate export/validation outputs. Implementations can be local, server-based, cloud-based, or distributed, with auditability and versioned candidate histories.

The disclosure is theory-compatible but not theory-limited. Broader scientific context may be used as rationale; practice of the invention does not require acceptance or proof of any single external theory. The claimed methods and systems are enabled by the disclosed computational rules, parameters, thresholds, module interactions, workflow stages, and validation pathways. Accordingly, this condensed Detailed Description preserves the same technical architecture: D=(S, H, E), thresholded candidate gating, structural compatibility evaluation, coherence selection, predicted enhancement-based ranking, simulator-based implementation, validation workflow, therapeutic deployment options, and computer-system support with figure-anchored reference numerals.

Worked example (illustrative ranking run). In an illustrative implementation, the system receives a target-associated candidate sequence and applies the stored counting convention to compute #CG and #AT and the stability score S_stab=1.5*(#CG)+1.0*(#AT). The system rejects candidates with S_stab<4.5 and advances candidates with S_stab>=4.5 to structural evaluation. For structural evaluation, the system selects a pitch parameter P (e.g., 32 angstroms) and samples r in [0, P] using a selected step size dr (e.g., dr within 0.1 to 1.0 angstroms), computes Dev as either max over r of abs(C_cand(r)−C_ref(r)) or an RMS deviation, and accepts candidates satisfying Dev<=0.006. For accepted candidates, the system selects a coherence level n from an allowed set (e.g., n in {1,2,3}), computes predicted enhancement as (S_stab/3)\hat{ }2*n, ranks candidates based on predicted enhancement (optionally using Dev as a tie-breaker), stores the stability, Dev, coherence, and predicted enhancement values in candidate records, and outputs a ranked results report for downstream synthesis and validation.

What is claimed is:

1. A computer-implemented method for selecting a candidate nucleic acid sequence for age-reversal-associated use, the method comprising:
receiving, by one or more processors, a target associated with a longevity-associated gene or a candidate nucleic acid sequence corresponding to the target;
computing, for the candidate nucleic acid sequence, a sequence stability score according to a scoring convention that computes a weighted sum of (i) a CG occurrence count and (ii) an AT occurrence count under a stored counting convention;
determining whether the sequence stability score satisfies a stability threshold;
for a candidate nucleic acid sequence that satisfies the stability threshold, evaluating structural compatibility of the candidate nucleic acid sequence according to a helical parameter set comprising a pitch parameter and a groove ratio parameter;
for a candidate nucleic acid sequence that satisfies the structural compatibility evaluation, selecting a coherence level for the candidate nucleic acid sequence from an allowed set of coherence levels;
computing, using the sequence stability score and the selected coherence level, a predicted enhancement score for the candidate nucleic acid sequence;
ranking the candidate nucleic acid sequence relative to one or more other candidate nucleic acid sequences based on at least the predicted enhancement score; and
selecting the candidate nucleic acid sequence for synthesis, construct assembly, editing, delivery, or validation based on the ranking.

2. The method of claim 1, wherein the stability threshold requires that the sequence stability score be at least 4.5.

3. The method of claim 1, wherein computing the sequence stability score comprises computing the sequence stability score according to:
$$S\_stab = 1.5 * ( \# CG ) + 1. * ( \# AT ) .$$

4. The method of claim 1, wherein evaluating structural compatibility comprises evaluating the candidate nucleic acid sequence using a pitch parameter in a range of 30 to 40 angstroms.

5. The method of claim 1, wherein evaluating structural compatibility comprises evaluating the candidate nucleic acid sequence using a groove ratio parameter in a range of 1.5 to 1.7.

6. The method of claim 1, wherein evaluating structural compatibility comprises: evaluating a structural compatibility function over an evaluation domain and determining whether the candidate nucleic acid sequence satisfies a tolerance criterion,
wherein evaluating the structural compatibility function comprises sampling r over r∈[0, P] using a step size Δr, generating a candidate-derived profile C_cand(r) from the candidate nucleic acid sequence by (i) computing a sequence-derived feature vector under the stored counting convention, (ii) mapping the sequence-derived feature vector to a local stiffness or curvature proxy using a stored mapping table, and (iii) interpolating the local stiffness or curvature proxy to generate a continuous profile over the evaluation domain, and wherein the tolerance criterion is based on a deviation metric Dev computed between C_cand(r) and a reference profile C_ref(r) over the evaluation domain.

7. The method of claim 6, wherein the tolerance criterion requires a structural deviation of no greater than 0.006 over the evaluation domain.

8. The method of claim 1, wherein selecting the coherence level comprises selecting n=2.

9. The method of claim 1, wherein computing the predicted enhancement score comprises computing the predicted enhancement score according to:
$$Predicted enhancement = ( S\_stab / 3 ) \hat{} 2 * n .$$

10. The method of claim 1, wherein the target is associated with at least one of SIRT1, FOXO3, TERT, XRCC1, RAD51, ATM, PARP1, or KLOTHO.

11. The method of claim 1, wherein the candidate nucleic acid sequence comprises a promoter sequence, an enhancer sequence, a coding sequence, a donor-template sequence, or a repair-template sequence.

12. The method of claim 1, wherein ranking the candidate nucleic acid sequence comprises selecting the candidate nucleic acid sequence as part of a top-N subset of candidate nucleic acid sequences, and applying a tie-break rule that favors a lower value of the structural deviation metric Dev when predicted enhancement scores are equal, and storing, in a candidate record, at least the sequence stability score, the selected coherence level, the predicted enhancement score, and the structural deviation metric Dev.

13. The method of claim 1, further comprising generating a validation plan for the selected candidate nucleic acid sequence, the validation plan comprising at least one of a reporter assay, a repair assay, a senescence assay, a lifespan assay, or an in vivo assessment.

14. A method of producing an age-reversal-associated biological effect, the method comprising:
obtaining a nucleic acid construct comprising a nucleic acid sequence selected by the method of claim 1; and
introducing the nucleic acid construct into a cell or administering the nucleic acid construct to a subject, thereby increasing expression or activity of a longevity-associated target, increasing repair-associated function, reducing a biological aging indicator, increasing cellular lifespan, or a combination thereof.

15. The method of claim 14, wherein the age-reversal-associated biological effect is confirmed by a measured improvement over a control of at least 20% in at least one of a reporter assay metric, a repair assay metric, a senescence assay metric, a lifespan assay metric, or an in vivo assessment metric.

16. The method of claim 14, wherein introducing the nucleic acid construct comprises introducing a donor template or editing construct configured for CRISPR-mediated editing of an endogenous locus.

17. The method of claim 14, wherein the nucleic acid construct comprises a multi-gene cassette comprising two or more elements associated with SIRT1, FOXO3, TERT, XRCC1, RAD51, ATM, PARP1, or KLOTHO.

18. A system for selecting a candidate nucleic acid sequence for age-reversal-associated use, the system comprising:
one or more processors; and memory storing instructions that, when executed by the one or more processors, cause the system to: receive a target associated with a longevity-associated gene or a candidate nucleic acid sequence corresponding to the target; compute, for the candidate nucleic acid sequence, a sequence stability score according to a scoring convention that computes a weighted sum of (i) a CG occurrence count and (ii) an AT occurrence count under a stored counting convention; determine whether the sequence stability score satisfies a stability threshold; for a candidate nucleic acid sequence that satisfies the stability threshold, evaluate structural compatibility of the candidate nucleic acid sequence according to a helical parameter set comprising a pitch parameter and a groove ratio parameter; for a candidate nucleic acid sequence that satisfies the structural compatibility evaluation, select a coherence level for the candidate nucleic acid sequence from an allowed set of coherence levels; compute, using the sequence stability score and the selected coherence level, a predicted enhancement score for the candidate nucleic acid sequence; rank the candidate nucleic acid sequence relative to one or more other candidate nucleic acid sequences based on at least the predicted enhancement score; and select the candidate nucleic acid sequence for synthesis, construct assembly, editing, delivery, or validation based on the ranking.

19. The system of claim 18, wherein the memory stores the counting convention and associates the counting convention with each candidate record in a candidate data store.

20. The system of claim 18, wherein the system computes structural deviation Dev between a candidate-derived profile C_cand(r) and a reference profile C_ref(r) by sampling r over r in [0, P] using a step size dr, and computing Dev as either (i) the maximum, over sampled r values, of abs(C_cand(r)−C_ref(r)) or (ii) an RMS deviation over the sampled domain, and wherein the system computes C_cand(r) from the candidate nucleic acid sequence by deriving a sequence-derived feature vector under the stored counting convention, mapping the feature vector using a stored mapping table to a local stiffness or curvature proxy, and interpolating the proxy to generate a continuous profile over the evaluation domain.
